(19) United States
(12) Patent Application Publication
(10) Pub. No.: US 2023/0074429 A1
(43) Pub. Date: Mar. 9, 2023

(54) RESPIRATION COMPENSATION

(71) Applicant: St. Jude Medical, Cardiology Division, Inc., St. Paul, MN (US)

(72) Inventors: Silvina Rybnikov, Zichron Ya'acov (IL); Adar Shlain, Hadar Am (IL); Alexander Zaslavsky, Haifa (IL)

(21) Appl. No.: 17/798,396

(22) PCT Filed: Dec. 31, 2020
(86) PCT No.: PCT/IB2020/062573

§ 371 Date: Aug. 9, 2022

Related U.S. Application Data
(60) Provisional application No. 62/972,370, filed on Feb. 10, 2020.

(51) Int. Cl. A61B 5/113; A61B 5/283; A61B 5/00

(52) U.S. Cl. CPC A61B 5/113; A61B 5/283; A61B 5/6858; A61B 5/721

(57) ABSTRACT

Embodiments of the present disclosure include a method for performing patient respiration compensation. The method can include receiving a position sensor signal from a position sensor disposed on a catheter. The method can include determining a position sensor location of the position sensor from the position sensor signal. The method can include receiving a patient reference sensor signal from a patient reference sensor. The method can include determining a patient reference sensor location of the patient reference sensor from the patient reference sensor signal. The method can include determining a weight for the patient reference sensor location. The method can include determining an average of the patient reference sensor location. The method can include determining a compensation for the patient respiration, based on the weighted patient reference sensor location and the average of the patient reference sensor location.

### BACKGROUND

### a. Field of the Invention

The present disclosure relates generally to respiration compensation.

### b. Background Art

Medical devices, catheters, and/or cardiovascular catheters, such as electrophysiology catheters can be used in a variety of diagnostic, therapeutic, mapping and/or ablative procedures to diagnose and/or correct conditions such as atrial arrhythmias, including for example, ectopic atrial tachycardia, atrial fibrillation, and atrial flutter. A medical device can be threaded through a vasculature of a patient to a site where the diagnostic, therapeutic, mapping, and/or ablative procedure to diagnose and/or correct the condition is performed.

Sensors (e.g., electrodes, magnetic positioning sensors) can be placed on the medical device, which can receive signals that are generated proximate to the patient from a device. Based on the received signals, an orientation and/or position of the medical device within a heart can be computed.

One technique for determining the position and orientation of a catheter within a body is by tracking a plurality of sensors on the catheter using a position sensing and navigation system (sometimes called a location mapping system). The sensors can include electrodes disposed on the catheter, which can provide voltage measurements associated with their exposure to an electrical field generated through excitation of pairs of electrodes on an outer surface of the body. Voltage measurements on the catheter electrodes can then be used to determine the position and orientation of the catheter electrodes within a coordinate system of the position sensing and navigation system. Other exemplary position sensing and navigation systems include magnetic systems. For example, a magnetic position sensor can be disposed on a catheter and a magnetic field can be generated by one or more magnetic field generators. Responsive to a particular location of the magnetic position sensor in the magnetic field, a position of the magnetic position sensor, and thus the catheter, can be determined.

In order to provide information to clinicians about the position and orientation of the catheter, the determined position and orientation of the catheter sensors can be used to render an image of the catheter relative to surrounding tissues, including heart tissues. One drawback to conventional systems, however, is that the determined position and orientation of the catheter sensors can include artifacts due to respiration of a patient. For example, as a patient breaths while laying on a patient examination table, the chest of the patient can rise and fall, as a result of respiration. This can cause a position of the position sensor (e.g., impedance or magnetic based) that is disposed on the catheter to change, due to the rising and falling of the patient's chest. As a result, even when the catheter is stationary with respect to a heart of a patient, the catheter can appear as it is moving (e.g., rising and falling), due to respiration of the patient.

### SUMMARY

Embodiments of the present disclosure include a method for performing patient respiration compensation. In some embodiments, the method can comprise receiving a position sensor signal from a position sensor disposed on a catheter. In some embodiments, the method can include determining a position sensor location of the position sensor from the position sensor signal. In some embodiments, the method can include receiving a patient reference sensor signal from a patient reference sensor. In some embodiments, the method can include determining a patient reference sensor location of the patient reference sensor from the patient reference sensor signal. In some embodiments, the method can include determining a weight for the patient reference sensor location, based on a comparison between a movement of the position sensor location and a movement of the patient reference sensor location. In some embodiments, the method can include determining an average of the patient reference sensor location. In some embodiments, the method can include determining a compensation for the patient respiration, based on the weighted patient reference sensor location and the average of the patient reference sensor location.

Embodiments of the present disclosure can include a method for performing patient respiration compensation. In some embodiments, the method can include receiving a position sensor signal from a position sensor disposed on a catheter. In some embodiments, the method can include determining a position sensor location of the position sensor from the position sensor signal. In some embodiments, the method can include receiving a plurality of electrode patch signals from a plurality of respective electrode patches disposed on a body of the patient. In some embodiments, the method can include determining a plurality of electrode patch locations of the plurality of electrode patches, from the plurality of electrode patch signals. In some embodiments, the method can include determining a plurality of respective weights for the plurality of electrode patch locations, based on a comparison between a movement of the position sensor location and a movement of each of the plurality of electrode patch locations. In some embodiments, the method can include determining a compensation for the patient respiration, based on the weighted electrode patch location.

Embodiments of the present disclosure include a system for performing patient respiration compensation. The system can include a computing device comprising processor resources and memory resources, the memory resources storing computer-readable instructions that, when executed by the processor resources, cause the processor resources to receive a position sensor signal from a position sensor disposed on a catheter. In some embodiments, the computer-readable instructions can be executed by the processor resources to determine a position sensor location of the position sensor from the position sensor signal. In some embodiments, the computer-readable instructions can be executed by the processor resources to receive an electrode patch signal from an electrode patch disposed on a body of the patient. In some embodiments, the computer-readable instructions can be executed by the processor resources to receive a patient reference sensor signal from a patient reference sensor. In some embodiments, the computer-readable instructions can be executed by the processor resources to determine an electrode patch location of the electrode patch, from the electrode patch signal. In some embodiments, the computer-readable instructions can be executed by the processor resources to determine a patient reference sensor location of the patient reference sensor from the patient reference sensor signal. In some embodiments, the computer-readable instructions can be executed by the processor resources to determine respective weights for the electrode patch location and the patient reference sensor location, based on a comparison between a movement of the position sensor location with respect to a movement of the electrode patch location and the patient reference sensor location. In some embodiments, the computer-readable instructions can be executed by the processor resources to determine a compensation for the patient respiration, based on the weighted electrode patch location and the weighted reference sensor location.

### BRIEF DESCRIPTION OF THE DRAWINGS

FIG. 1 is a diagrammatic overview of a catheter system, in accordance with embodiments of the present disclosure.

FIG. 2A depicts an isometric side view of an electrophysiology catheter, in accordance with embodiments of the present disclosure.

FIG. 2B depicts a top view of a second type of electrophysiology catheter, in accordance with embodiments of the present disclosure.

FIG. 3A depicts an anterior view of a patient disposed on a patient examination table, in accordance with embodiments of the present disclosure.

FIG. 3B depicts a posterior view of the patient, in accordance with embodiments of the present disclosure.

FIG. 4 depicts a method flow diagram for performing patient respiration compensation, in accordance with embodiments of the present disclosure.

FIG. 5 depicts an alternate method flow diagram for performing patient respiration compensation, in accordance with embodiments of the present disclosure.

FIG. 6 depicts a system for performing patient respiration compensation, in accordance with embodiments of the present disclosure.

### DETAILED DESCRIPTION

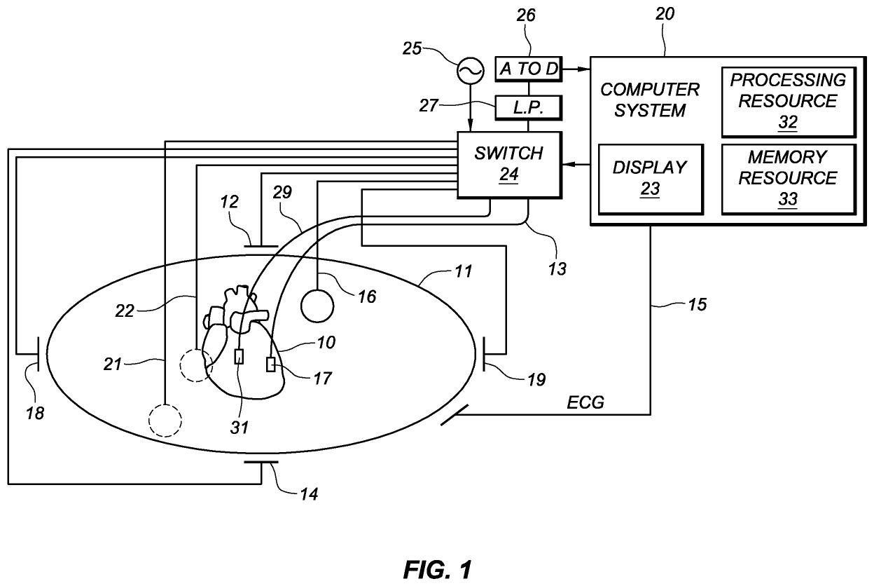

Referring now to the drawings wherein like reference numerals are used to identify identical components in the various views, FIG. 1 is a diagrammatic overview of a catheter system in which the invention may be practiced. The system may comprise various visualization, mapping and navigation components as known in the art, including among others, for example, an EnSite™ Precision™ Cardiac Mapping and Visualization System commercially available from Abbott Laboratories, or as seen generally by reference to U.S. Pat. No. 7,263,397, owned by the common assignee of the present invention, and hereby incorporated by reference in its entirety.

The system may be used in connection with or for various medical procedures, for example, mapping of the heart and/or cardiac ablation procedures. In one embodiment, the medical positioning system 14 may comprise a magnetic field-based system such as, for example, the Carto™ System available from Biosense Webster, and as generally shown with reference to one or more of U.S. Pat. Nos. 6,498,944; 6,788,967; and 6,690,963, the entire disclosures of which are incorporated herein by reference, or the MediGuide™ system from MediGuide Ltd. (now owned by Abbott Laboratories), and as generally shown with reference to one or more of U.S. Pat. Nos. 6,233,476; 7,197,354; and 7,386,339, the entire disclosures of which are incorporated herein by reference. Alternatively, the medical positioning system 14 may comprise a combination of a magnetic field-based system and an electric field-based system such as, for example and without limitation, the Carto 3™ System also available from Biosense Webster. Although reference is made to cardiac mapping of the heart, one or more aspects of the present disclosure may apply to other anatomic structures.

With reference to FIG. 1, the catheter system includes a diagrammatic depiction of a heart 10 of a patient 11. The system includes the ability to receive a plurality of catheter locations as a catheter distal end is swept around and within a chamber of the heart. For this purpose, FIG. 1 shows an exemplary catheter localization system of the type based on externally-applied orthogonal electric fields, which are used to determine the location of one or more catheter position sensors. An example of such a system is an EnSite NAVX™ Navigation and Visualization System. It should be understood, however, that this embodiment is exemplary only and not limiting in nature. Other technologies for determining the location in 3D space of a catheter, such as the MediGuide™ system, may be used in practicing the present invention, including for example, the CARTO navigation and location system of Biosense Webster, Inc., or the AURORA® system of Northern Digital Inc., both of which utilize magnetic fields rather than electrical fields. Accordingly, as used herein, a sensor is provided for producing signals indicative of catheter location information, and may include one or more position sensors. The position sensors can include one or more electrodes configured to detect one or more characteristics of an electrical field, for example in the case of an impedance-based localization system, or alternatively, one or more coils (e.g., wire windings) configured to detect one or more characteristics of a magnetic field, for example, in the case of a magnetic-field based localization system.

It should be further understood that in some localization systems, one or more position sensors may collectively define the sensor. The one or more position sensors may be provided on a distal end of a catheter and the localization system may be configured to obtain location information from the one or more position sensors. The localization system may compute a distal location of the catheter using not only the received location information, but also a geometrical relationship between the one or more position sensors providing the location information and the distal location on the catheter (e.g., one piece of geometrical information may be the ring electrode to tip distance). Finally, the localization system may use the computed location, as if it were collected directly. Likewise, in a magnetic field based localization embodiment, the catheter tip and the magnetic coil may have a geometric relationship therebetween where the localization system is configured to use the computed tip location (i.e., computed based on the magnetic coil signals and predefined knowledge of the geometrical relationship between coil and tip) as if such location were collected directly. Of course, other variations are possible.

With continued reference to FIG. 1, in the illustrated impedance-based localization system embodiment, three sets of surface electrodes (e.g., applied via a patch) are shown: X-axis electrodes 12, 14; Y-axis electrodes 18, 19; and Z-axis electrodes 16, 22. Additionally, an additional surface electrode 21 (e.g., applied via a “belly” patch) may be used. The surface electrodes are all connected to a switch 24. A representative catheter 13 is shown, which has a single distal electrode 17, which may be referred to herein as a “roving” or “measurement” electrode. In some embodiments, the catheter 13 can be a coronary sinus catheter or a right ventricle apex catheter. The electrode 17 may define the position sensor in this embodiment, but as alluded to above, many variations are possible and the catheter 13 can include multiple position sensors, as discussed further herein. FIG. 1 also shows a second, independent catheter 29 with a fixed reference electrode 31, which may be stationary on the heart 10 for calibration purposes.

Although a catheter is generally discussed herein for use with embodiments of the present disclosure, embodiments of the present disclosure are not so limited and can be used with other types of medical devices. For example, embodiments of the present disclosure can be used with introducers, sheaths, among other types of medical devices, where it may be desirable to account for patient respiration when determining a location of the medical device.

FIG. 1 further shows a computer system 20, a signal generator 25, an analog-to-digital converter 26 and a low-pass filter 27. The computer system 20 can utilize software, hardware, firmware, and/or logic to perform a number of functions described herein. The computing system 20 can be a combination of hardware and instructions to share information. The hardware, for example can include processing resource 32 and/or a memory resource 33 (e.g., non-transitory computer-readable medium (CRM) database, etc.). A processing resource 32, as used herein, can include a number of processors capable of executing instructions stored by the memory resource 33. Processing resource 32 can be integrated in a single device or distributed across multiple devices. The instructions (e.g., computer-readable instructions (CRI)) can include instructions stored on the memory resource 33 and executable by the processing resource 32 for aligning a cardiac model.

The computer system 20 is configured to control the signal generator 25 in accordance with predetermined strategies to selectively energize various pairs of surface electrodes. In operation, the computer system 20 is configured to obtain raw patch data (i.e., voltage readings) via the filter 27 and A-D converter 26 and use this raw patch data to determine the raw electrode location coordinates in three-dimensional space (X, Y, Z) of a catheter electrode positioned inside the heart 10 or chamber thereof (e.g., such as the roving electrode 17 mentioned above). In some embodiments, a phase of the patient's 11 cardiac cycle can be measured or otherwise determined when such electrode location coordinates are being received. For this purpose, in an embodiment, most or all of the conventional twelve (12) ECG leads, coupled to body surface electrodes and designated collectively by reference numeral 15, are provided to support the acquisition of an electrocardiogram (ECG) of the patient 11.

Alternatively, a reference electrode positioned in a fixed location in the heart 10, such as fixed reference electrode 31, may be used to provide a relatively stable signal that can be analyzed to determine the cardiac phase of the heart 10 in the cardiac cycle (e.g., placed at the coronary sinus). More generally, another catheter having an electrode, other than the moving or roving catheter, may be placed and maintained in a constant position relative to the heart 10 to obtain a relatively stable signal indicative of cardiac phase. As shown, the ECG leads 15 are coupled directly to the computer system 20 for acquisition and subsequent processing to obtain the phase of the heart 10 in the cardiac cycle. The ECG leads 15 may also be provided to other systems (not shown).

As previously mentioned, embodiments of the present disclosure can be used with a magnetic field-based system. Some embodiments can include a main electronic control unit (e.g., one or more processors) having various input/output mechanisms, a display, an optional image database, a localization system such as a medical positioning system (MPS) (electromagnetic sensor tracking system), an electrocardiogram (ECG) monitor, one or more MPS location sensors (e.g., patient reference sensor), and an MPS-enabled medical device (such as an elongated catheter or introducer) which itself includes one or more of the above-described MPS location sensors. As discussed, in some embodiments, the medical positioning system may comprise a magnetic field-based system such as, for example, the MediGuide™ system from MediGuide Ltd. (now owned by Abbott Laboratories), and as generally shown with reference to one or more of U.S. Pat. Nos. 6,233,476; 7,197,354; and 7,386,339, the entire disclosures of which are incorporated herein by reference.

Embodiments can include input/output mechanisms, which can comprise conventional apparatus for interfacing with a computer-based control unit, for example, a keyboard, a mouse, a tablet, a foot pedal, a switch or the like. Embodiments can also include a display, which can also comprise conventional apparatus.

Embodiments may find use in navigation applications that use imaging of a region of interest. Therefore, the magnetic field-based system may optionally include an image database. The image database may be configured to store image information relating to the patient's body, for example, a region of interest surrounding a destination site for the medical device and/or multiple regions of interest along a navigation path contemplated to be traversed by the device to reach the destination site. The image data in the image database may comprise known image types including (1) one or more two-dimensional still images acquired at respective, individual times in the past; (2) a plurality of related two-dimensional images obtained in real-time from an image acquisition device (e.g., fluoroscopic images from an x-ray imaging apparatus) wherein the image database acts as a buffer (live fluoroscopy); and/or (3) a sequence of related two-dimensional images defining a cine-loop (CL), wherein each image in the sequence has at least an ECG timing parameter associated therewith adequate to allow playback of the sequence in accordance with acquired real-time ECG signals obtained from the ECG monitor. It should be understood that the foregoing are examples only and not limiting in nature. For example, the image database may also include three-dimensional image data. It should be further understood that the images may be acquired through any imaging modality, now known or hereafter developed, for example X-ray, ultra-sound, computerized tomography, nuclear magnetic resonance or the like.

The MPS can be configured to serve as the localization system and therefore to determine positioning (localization) data with respect to one or more of MPS location sensors, one or more medical devices, and/or one or more patient reference sensors (PRS), and output a respective location reading. The location readings may each include at least one or both of a position and an orientation (P&O) relative to a reference coordinate system, which may be the coordinate system of the MPS. For example, the P&O may be expressed as a position (i.e., a coordinate in three axes X, Y, and Z) and orientation (i.e., an azimuth and elevation) of a magnetic field sensor in a magnetic field relative to a magnetic field generator(s) or transmitter(s).

The MPS determines respective locations (i.e., P&O) in the reference coordinate system, based on capturing and processing signals received from the magnetic field sensors, while such sensors are disposed in a controlled low-strength AC magnetic field. From an electromagnetic perspective, these sensors develop a voltage that is induced on the coil residing in a changing magnetic field, as contemplated here. The sensors are thus configured to detect one or more characteristics of the magnetic field(s) in which they are disposed and to generate an indicative signal, which is further processed by the MPS to obtain a respective P&O of the sensors. Exemplary design features and manufacturing processes and methods for the sensors and medical devices incorporating such sensors may be found in U.S. Pat. No. 8,636,718, the entirety of which is incorporated by reference herein.

The MPS sensor, and optionally additional MPS sensors in further embodiments, may be associated with the MPS-enabled medical device. Another MPS sensor, namely, a patient reference sensor (PRS) is configured to provide a positional reference of the patient's body so as to allow motion compensation for gross patient body movements and/or respiration-induced movements. The PRS may be attached to the patient's manubrium sternum, a stable place on the chest, or another location that is relatively positionally stable. Like MPS location sensor, the PRS is configured to detect one or more characteristics of the magnetic field in which it is disposed, wherein the MPS provides a location reading (e.g., a P&O reading) indicative of the PRS's position and orientation in the reference coordinate system.

The electro-cardiogram (ECG) monitor is configured to continuously detect an electrical timing signal of the heart organ through the use of a plurality of ECG electrodes (not shown), which may be externally-affixed to the outside of a patient's body. The timing signal generally corresponds to the particular phase of the cardiac cycle, among other things. Generally, the ECG signal(s) may be used by the control unit for ECG synchronized playback of a previously captured sequence of images (cine loop) stored in the database. The ECG monitor and the ECG-electrodes may both comprise conventional components.

The magnetic field-based system can be incorporated into or associated with a fluoroscopic imaging system, which may include commercially available fluoroscopic imaging components, for example, an x-ray source, a C-Arm, and/or an x-ray image intensifier or detector (i.e., “Catheter Lab”). The MPS (electromagnetic sensor tracking system) includes a magnetic transmitter assembly (MTA) (electromagnetic field generator) and a magnetic processing core for determining location (P&O) readings. The MTA is configured to generate the magnetic field(s) in and around the patient's chest cavity, in a predefined three-dimensional space identified as a motion box.

The MPS sensors are, as described above, configured to sense one or more characteristics of the magnetic field(s) when the sensors are in a motion box, and each generate a respective signal that is provided to the magnetic processing core. The processing core is responsive to these detected signals and is configured to calculate respective P&O readings for each MPS sensor in the motion box. The processing core can detect when an MPS sensor exits the motion box. Thus, the MPS enables real-time tracking of each sensor in three-dimensional space.

The actual volume of the motion box may be stored in, for example, the processing core, and processing core is able to determine the positions and orientations of each sensor in relation to the boundaries of the motion box. Alternatively, the actual volume of motion box may be stored in, for example, the main control, and the main control may be able to determine the positions and orientations of each sensor in relation to the boundaries of the motion box. Accordingly, the system can evaluate (e.g., in the processing core or in the main control) whether a sensor is within, at the boundary of, or outside of the motion box. Based on this information, the motion box and sensor(s) can be displayed in relation to one another on the display as described in greater detail elsewhere herein.

In some alternative embodiments, the MTA can be located underneath a patient examination table, between an x-ray source and the patient examination table. For example, the MTA can be connected with the patient examination table. In some embodiments, as discussed herein, the MTA can be a mobile device, which can be placed on a chest of the patient and used to generate the magnetic field for tracking of the object.

In those embodiments in which MPS sensors are used in conjunction with an imaging system, the positional relationship between the image coordinate system and the MPS reference coordinate system (electromagnetic tracking coordinate system) may be calculated based on a known optical-magnetic calibration of the system (e.g., established during setup), since the positioning system and imaging system may be considered as being fixed relative to each other in such an embodiment. However, for other embodiments using other imaging modalities, including embodiments where the image data is acquired at an earlier time and then imported from an external source (e.g., imaging data stored in the database), a registration step registering the MPS coordinate system and the image coordinate system may need to be performed so that MPS location readings can be properly coordinated with any particular image being used.

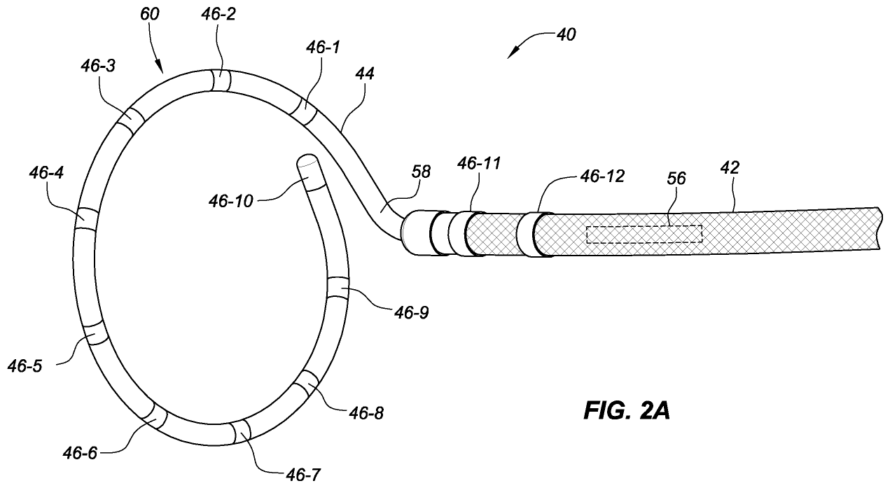

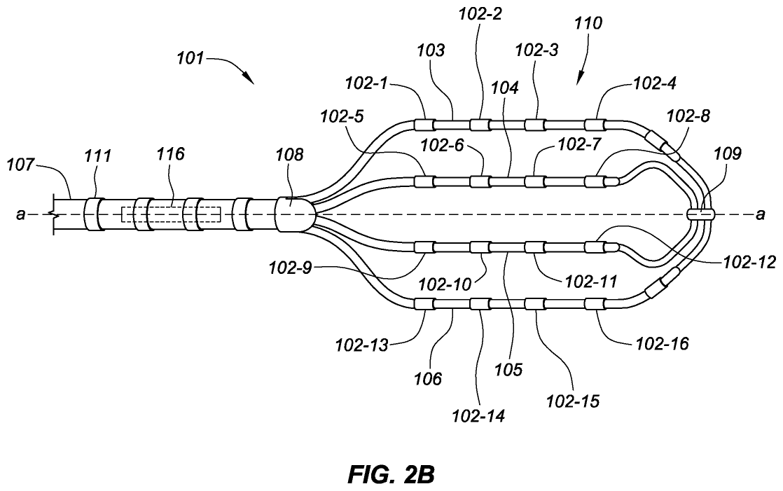

FIG. 2A depicts an electrophysiology catheter 40, in accordance with embodiments of the present disclosure. The electrophysiology catheter 40 can be used in an electrophysiology procedure to help doctors understand a nature of abnormal heart rhythms (e.g., arrhythmias) and can include features as further discussed in WO 2017/177121, which is hereby incorporated by reference as though fully set forth herein. The electrophysiology procedure is performed by inserting the electrophysiology catheter 40, which measures electrical activity, through blood vessels that enter the heart. Each electrophysiology catheter 40 can include several electrodes 46-1, 46-2, . . . , 46-12 connected to a computer system (e.g., computer system 20 in FIG. 1) via a connection box. Hereinafter, the electrodes 46-1, 46-2, . . . , 46-12 are referred to in the plural as electrodes 46. The electrodes 46 can be disposed on a flexible tip portion 44, which can be a circular tip, as depicted. However, the tip can be formed as other shapes, in some embodiments.

The electrophysiology catheter 40 can include a magnetic position sensor 56, in some embodiments, disposed in and/or on the shaft 42. The electrodes can detect one or more characteristics of an electrical field in which the electrodes 46 are disposed. As previously discussed herein, in relation to FIG. 1, the electrical field can be produced by surface electrodes (e.g., patch electrodes) placed on an exterior of the patient. Based on the impedances associated with signals received from the electrodes 46, a position (e.g., coordinates) of the electrophysiology catheter 40 can be determined. In some embodiments, the electrophysiology catheter 40 can be an Advisor™ FL Circular Mapping Catheter, Sensor Enabled™, as produced by Abbott Laboratories, although the electrophysiology catheter 40 can be another type of electrophysiology catheter in some embodiments. The catheter 40 can be used in conjunction with an EnSite™ Precision Cardiac Mapping and Visualization System and/or a MediGuide™ system, among other types of systems, for example those mentioned herein.

FIG. 2B is a top view of a second type of electrophysiology catheter 101, in accordance with embodiments of the present disclosure. The electrophysiology catheter is also referred to herein as a high density electrode catheter 101. In some embodiments, the high density electrode catheter 101 can include a flexible tip portion 110 that forms a flexible array of microelectrodes 102. This planar array (or ‘paddle’ configuration) of microelectrodes 102 comprises four side-by-side, longitudinally-extending arms 103, 104, 105, 106, which can form a flexible framework on which the microelectrodes 102 are disposed. The four microelectrode-carrier arms can comprise a first outboard arm 103, a second outboard arm 106, a first inboard arm 104, and a second inboard arm 105, which can be joined at a distal end by a distal connective portion 109, although not required. These arms can be laterally separated from each other.

Each of the four arms can carry a plurality of microelectrodes 102. For example, each of the four arms can carry microelectrodes 102 spaced along a length of each of the four arms. Although the high density electrode catheter 101 depicted in FIG. 2B depicts four arms, the high density electrode catheter 101 could comprise more or fewer arms. Additionally, while the high density electrode catheter 101 depicted in FIG. 2B depicts 18 electrodes (e.g., 5 microelectrodes on the first outboard arm 103 and second outboard arm 106, and 4 microelectrodes on the first inboard arm 104 and second inboard arm 105), the catheter can include more or fewer than 18 electrodes. In addition, the first outboard arm 103 and second outboard arm 106 can include more or fewer than 5 microelectrodes and the first inboard arm 104 and second inboard arm 105 can include more or fewer than 4 microelectrodes).

In some embodiments, the microelectrodes 102 can be used in diagnostic, therapeutic, and/or mapping procedures. For example and without limitation, the microelectrodes 102 can be used for electrophysiological studies, pacing, cardiac mapping, and ablation. In some embodiments, the microelectrodes 102 can be used to perform unipolar or bipolar ablation. This unipolar or bipolar ablation can create specific lines or patterns of lesions. In some embodiments, the microelectrodes 102 can receive electrical signals from the heart, which can be used for electrophysiological studies. In some embodiments, the microelectrodes 102 can perform a location or position sensing function related to cardiac mapping.

In some embodiments, the high density electrode catheter 101 can include a catheter shaft 107. The catheter shaft 107 can include a proximal end and a distal end. The distal end can include a connector 108, which can couple the distal end of the catheter shaft 107 to a proximal end of the planar array. The catheter shaft 107 can define a catheter shaft longitudinal axis aa, as depicted in FIG. 1A, along which the first outboard arm 103, first inboard arm 104, second inboard arm 105, and second outboard arm 106 can generally extend parallel in relation therewith. The catheter shaft 107 can be made of a flexible material, such that it can be threaded through a tortuous vasculature of a patient. In some embodiments, the catheter shaft 107 can include one or more ring electrodes 111 disposed along a length of the catheter shaft 107 and one or more magnetic position sensors 116 located in or along the shaft 107. The ring electrodes 111 can be used for diagnostic, therapeutic, and/or mapping procedures, in an example.

Embodiments of the present disclosure can provide for respiration compensation for a catheter location using impedance and magnetic positioning data. In an example, as a patient breathes on a patient examination table, a chest of the patient can experience movement due to respiration. As such, devices disposed inside of the patient's chest, such as a catheter in a heart of the patient can also experience movement due to respiration. For example, although a catheter may be held in fixed position with respect to a patient's heart, the catheter can still experience movement due to the patient's chest rising and falling, due to respiration.

When a geometric model of the heart is calculated, the model can depict that the catheter is moving, as a result of the catheter being moved from respiration. Embodiments of the present disclosure can combine data received from a magnetic patient reference sensor with impedance data received from electrode patches to improve respiration compensation. In an example, magnetic data received from the magnetic patient reference sensor can be more accurate than impedance data received from electrode patches, so the resulting respiration compensation is more reliable. When magnetic data becomes unavailable, for example, due to a magnetic disturbance (e.g., metallic distortion), embodiments of the present disclosure can revert to compensation based on electrode patches, only.

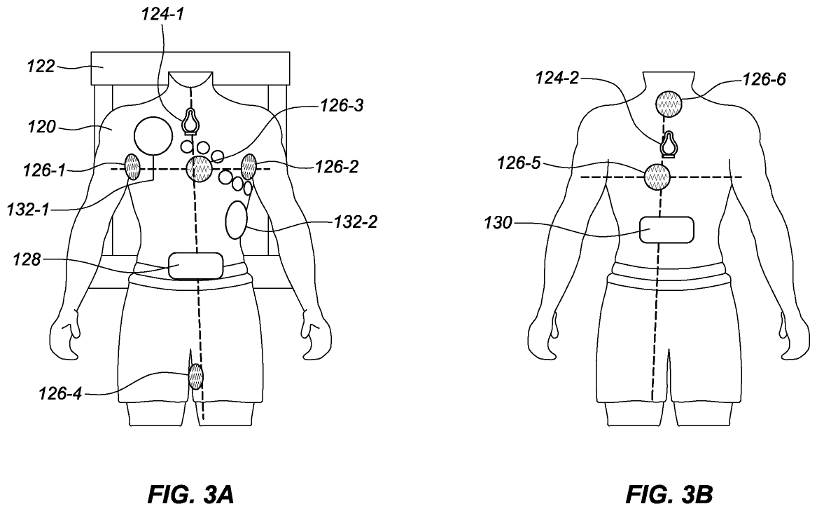

FIG. 3A depicts an anterior view of a patient 120 disposed on a patient examination table 122, in accordance with embodiments of the present disclosure. FIG. 3B depicts a posterior view of the patient 120, in accordance with embodiments of the present disclosure. As depicted in FIG. 3A, the patient is laying with their back disposed against the patient examination table 122 and has a plurality of electrodes, sensors, and other devices disposed on the patient's body. A first patient reference sensor 124-1 is disposed on a chest of the patient (e.g., anteriorly), as depicted in FIG. 3A, and a second patient reference sensor 124-2 is disposed on a back of the patient (e.g., posteriorly), as depicted in FIG. 3B. The patient reference sensors 124-1, 124-2 can be magnetic patient reference sensors, in some embodiments, and can produce a signal in response to being disposed in a magnetic field of varying intensity. The signal can be responsive to a position of the patient reference sensors 1204-1, 124-2 in the magnetic field. Using the signal, position coordinates of the patient reference sensors 124-1, 124-2 can be determined (e.g., using processing resources 32, depicted in FIG. 1). In some embodiments, as a patient breathes, position coordinates of the patient reference sensors 124-1, 124-2 can be used to account for the patient respiration, as further discussed herein.

As further depicted, a plurality of electrode patches 126-1, 126-2, . . . , 126-6 can be disposed anteriorly and posteriorly on the patient 120. Hereinafter, the electrode patches are referred to in the plural as electrode patches 126. In some embodiments, a left electrode patch 126-1 can be disposed on the patient's 120 left chest; a right electrode patch 126-2 can be disposed on the patient's 120 right chest; a front electrode patch 126-3 can be disposed on the patient's 120 center chest; a leg electrode patch 126-4 can be disposed on the patient's 120 leg; a back electrode patch 126-5 can be disposed on the patient's 120 center back; and a neck electrode patch 126-6 can be disposed on the patient's 120 neck. In some embodiments, an electrical field can be generated between the patches 126, which can be sensed by one or more electrodes disposed on a catheter inserted into the patient. Based on a signal received from the one or more electrodes disposed on the catheter, position coordinates of the catheter can be determined. In some embodiments, position coordinates of the electrode patches 126 can be determined, as a result of a signal generated by one of the electrode patches 126, responsive to a position of the one of the electrode patches 126 in the electrical field generated by the other electrode patches 126.

In some embodiments, a surface reference electrode 128 can be placed on a lower abdomen of the patient 120. In an example, the surface reference electrode 128 can provide a reference for other electrode patches 126. When an impedance based system measures impedance, it can measure the voltage (e.g., electric potential difference), and then use Ohm's law to calculate the impedance. The electric potential difference can be between two points, for example, such as a catheter electrode and the surface reference electrode 128 or the electrode patch 126 and the surface reference electrode 128. Thus, the surface reference electrode 128 can be used in impedance measurements made with the impedance based system.

In some embodiments, a radiofrequency (RF) dispersive patch 130 can be disposed on the patient's body 120. In an example where RF ablation is performed, a current can be passed between an ablation electrode disposed on the catheter and the RF dispersive patch 130. A surface area of the RF dispersive patch can be greater than a surface area of the ablation electrode, which can mitigate any heat buildup with respect to the dispersive patch. In some embodiments, a pair of defibrillator patches 132-1, 132-2 can be disposed on a chest of the patient. The defibrillator patches 132-1, 132-2 can be used for pacing of the patient's 120 heart, during a diagnostic and/or therapeutic procedure.

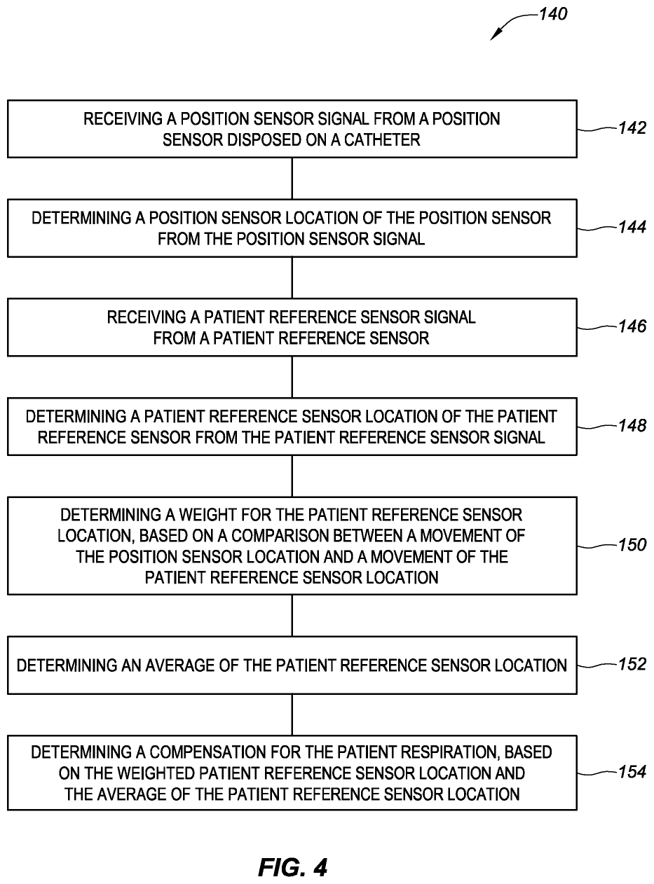

FIG. 4 depicts a method flow diagram 140 for performing patient respiration compensation, in accordance with embodiments of the present disclosure. In some embodiments, the method can be used in conjunction with a catheter that has been disposed inside of a patient. The catheter can include one or more position sensors disposed on the catheter, which can be used to determine a position of the catheter. The one or more position sensors disposed on the catheter can be magnetically and/or electrode based.

In some embodiments, a physician can insert the catheter into the patient's body. The catheter coordinates can be determined through the one or more position sensors disposed on the catheter and can be monitored (e.g., periodically or continually), using either impedance measurements for the catheter electrodes and/or magnetic measurements for the magnetic sensor/sensors mounted on the catheter. In some embodiments, a combination of the above two types of measurements can be used in determining the catheter coordinates. In such an embodiment, a filter (e.g., Extended Kalman Filter) can be used to filter the combination of the two types of measurements.

In some embodiments, a number of skin patches can be attached to the patient's body. In some embodiments, electrode patches can be disposed on the patient's chest, back, left and right armpits, neck, and/or left leg, however electrode patches can be disposed on other areas of the patient. In some embodiments, six electrode patches can be disposed on the patient's chest, back, left and right armpits, neck, and left leg. For each of these patches, the system can acquire a number of impedance signals corresponding to the different directions of the electric current induced in the body, for example, from back to chest, etc. In some embodiments, one or more patient reference sensors can be attached to the patient's body. For example, the one or more patient reference sensors can be attached to the patient's chest and/or to the patient's back. In some embodiments, magnetic position coordinates can be obtained from the patient reference sensors, which can be used to determine a location of the patient reference sensors.

As previously mentioned, when a patient breathes, a respiration artifact can be introduced with respect to position measurements taken by the position sensor disposed on the catheter. Embodiments of the present disclosure can offset the respiration artifact that is introduced by the patient respiration.

In some embodiments, the method 140 can include receiving 142 a position sensor signal from a position sensor disposed on a catheter. As discussed, the position sensor can be magnetic and/or electrode (e.g., impedance) based. In some embodiments, the catheter can include at least one magnetic based position sensor and at least one electrode based position sensor. The method 140 can include determining 144 a position sensor location of the position sensor from the position sensor signal. For example, with respect to a magnetically based position sensor, the position sensor location can be determined in relation to a signal produced by the magnetic position sensor, as a result of being disposed in a magnetic field with varying intensity. With respect to an electrode based position sensor, the position sensor location can be determined in relation to a signal produced by the electrode, as a result of being disposed in an electrical field.

In some embodiments, the method 140 can include receiving 146 a patient reference sensor signal from a patient reference sensor. In some embodiments, receiving the patient reference signal from the patient reference sensor includes receiving the patient reference signal from a magnetic patient reference sensor. In some embodiments, a first patient reference sensor can be disposed on a back of the patient (e.g., posteriorly) and a second patient reference sensor can be disposed on a chest of the patient (e.g., anteriorly). Each of the patient reference sensors can be disposed in a magnetic field of varying intensity and can produce a signal, responsive to a position of the patient reference sensor in the magnetic field. Using the signal, position coordinates of the patient reference sensor can be determined (e.g., using processing resources 32, depicted in FIG. 1).

In some embodiments, the method 140 can include determining 148 a patient reference sensor location of the patient reference sensor from the patient reference sensor signal. As discussed herein, the patient reference sensor can be disposed in a magnetic field and can generate a signal representative of a location of the patient reference sensor in the magnetic field. As a patient breathes, a distance between the first patient reference sensor and the second patient reference sensor can vary. Accordingly, respiration of the patient can be accounted for via the spatial relationship between the first patient reference sensor and the second patient reference sensor. In some embodiments, as discussed herein, the patient reference sensor can be attached externally to the patient.

In some embodiments, the method 140 can include determining 150 a weight for the patient reference sensor location, based on a comparison between a movement of the position sensor location and a movement of the patient reference sensor location. A compensation for patient respiration can then be determined based on the weighted patient reference sensor location. In some embodiments, the method can include determining a weight for more than one patient reference sensor location. For example, the method can include receiving a second patient reference sensor signal from a second patient reference sensor. In some embodiments, the second patient reference sensor can be a patient reference sensor that is placed on the patient to sense patient respiration. For example, the second patient reference sensor can be placed anteriorly or posteriorly on the patient to sense respiration.

With further reference to determining the weight for the patient reference sensor location, in some embodiments, the weight can be increased or decreased, based on a correlation in movement between the patient sensor location and the patient reference sensor location. For example, as a movement in the location of the patient sensor location and the patient reference sensor location more closely track one another, a correlation in movement can be defined as increasing. Furthermore, as a difference increases between the movement in the location of the patient sensor location and the patient reference sensor location, a correlation in movement can be defined as decreasing. In some embodiments, the weight for the patient reference sensor location can be increased, as a correlation in the movement between the patient sensor location and the patient reference sensor location increases. For example, as a correlation in movement between the patient sensor and the patient reference sensor increases, this can be an indication that the movement of the two sensors closely track one another. As such, a greater weight can be assigned to the patient reference sensor location as a correlation between the locations of the patient sensor and the patient reference sensor increase. Increasing a weight assigned to the patient reference sensor location can increase an effect that the patient reference sensor location has when determining the compensation for patient respiration.

In contrast, in some embodiments, the weight for the patient reference sensor location can be decreased, as a correlation in movement between the patient sensor location and the patient reference sensor location decreases. For example, as a correlation in movement between the patient sensor and the patient reference sensor decreases, this can be an indication that the movement of the two sensors do not closely track one another. As such, a lesser weight can be assigned to the patient reference sensor location as a correlation between the locations of the patient sensor and the patient reference sensor decreases. Decreasing a weight assigned to the patient reference sensor location can decrease an effect that the patient reference sensor location has when determining the compensation for patient respiration.

In some embodiments, the method can include determining a second patient reference sensor location of the second patient reference sensor from the second patient reference sensor signal. A second weight for the second patient reference sensor location can be determined based on a comparison between a movement of the position sensor location and a movement of the second patient reference sensor location. As discussed, the second weight can be determined based on a correlation in the movement between the position sensor location and the patient reference sensor location. As such, an effect that the location of the first patient reference sensor and the second patient reference sensor and other patient reference sensors, where additional patient reference sensors are used, can be tuned based on the weight assigned to each patient reference sensor location. For example, where the first patient reference sensor location and/or movement of the first patient reference sensor more closely resembles the location and/or movement of the position sensor than the second patient reference sensor, a greater weight can be assigned to the first patient reference sensor. Accordingly, the compensation for patient respiration can be determined based on the second weighted patient reference sensor location.

In an example, with respect to an x-coordinate of one of the catheter electrodes, X can be set as the x-coordinate and expressed as follows:

X=(Xᵢ),

where X is the x-coordinate of one of the catheter electrodes and i is a sample index. Although calculation of the compensation for patient respiration is illustrated herein for x-coordinates, the compensation for patient respiration can be determined in a same way for y-coordinates and z-coordinates. The compensation for patient respiration

C=(Cᵢ)

can be constructed such that

X−C

is as constant as possible when the physician does not move the catheter. If the location of the catheter does not move when the physician does not move the catheter, it can be indicative that the compensation for patient respiration C is effective. In an example, if artifacts from patient respiration are present, the catheter can appear to move, even though a physician is not moving the catheter. The movement can be due to the chest of the patient moving up and down, as a result of the patient respiration. When a graphical representation of the heart is created, the movement can be propagated in the graphical representation, causing the catheter to appear as though it is moving.

In some embodiments, the compensation for patient respiration can be constructed as a function of the patient reference sensor signals. For example, in a case where a first patient reference sensor is disposed on a first side of a patient (e.g., anteriorly) and a second patient reference sensor is disposed on a second side of the patient (e.g., posteriorly), a patient reference monitor signal

p=(pᵢ)

can be defined as

PRS-A(Z)−PRS-P(Z)

where Z is a back-to-chest coordinate axis of the patient reference sensors (PRS). The compensation can be defined by the equation:

C=α*m

where C is the compensation to be subtracted from the catheter location to offset the respiration artifact and m is defined as

m=p−pₐᵥₑᵣ,

which is a zero-average of the magnetic patient reference sensor signal, and a is the weight that is calculated for the patient reference sensor signal, as previously discussed.

The method 140 can include determining 152 an average of the patient reference sensor location. In some embodiments, the average of the patient reference sensor location can be a zero average. The zero average for the patient reference sensor signal can be determined when one or more patient reference sensor signals are obtained from one or more patient reference sensors. In an example, the zero average for the patient reference sensor(s) can be an average location of one or more of the patient reference sensors and/or an average movement (e.g., average track) of the one or more patient reference sensors. In some embodiments, the zero average can be determined over a period of time, such that the movements of the patient reference sensor having occurred due to respiration can be averaged into a single average track that can be used in determining the compensation for patient respiration. The zero average can be determined over a period of time that ranges up to 20 seconds. In some embodiments, the weight can be calculated for the patient reference sensor location, which is calculated from the patient reference sensor signal. Although embodiments of the present disclosure discuss determining a weight and/or average for a location and/or movement of a sensor, a weight and/or average can be determined for a signal received from a sensor.

In the above expression, m is a vector, where

m=(mᵢ)

with i being the sample index. The coefficient α can be calculated as a solution of the optimization problem

Σᵢ(xᵢ−αmᵢ)²→min

where x is defined as

x=X−Xₐᵥₑᵣ

and X is a zero average coordinate, which can be expressed in a same way for axes Y and Z. In the above expression, x is a vector

x=(xᵢ)

The solution of the above optimization problem can be provided by

$$α = {\sum }_{i} {x}_{i} {m}_{i} / {\sum }_{i} {m}_{i}^{2}$$

In the above referenced embodiments, the coefficients α and pₐᵥₑᵣ can be calculated over a learning period of a particular amount of time. In some embodiments, the learning period can include an amount of time in a range from 10 seconds to 15 seconds. In some embodiments, the learning period can be set to 12 seconds.

In some embodiments, the method 140 can include determining 154 a compensation for the patient respiration, based on the weighted patient reference sensor location and the average of the patient reference sensor location, as discussed above. For example, the compensation for patient respiration can be the product of the coefficient α and the zero-average magnetic patient reference sensor signal, which can be used to calculate the location and/or movement of the patient reference sensor, as discussed above. In some embodiments, the compensation for patient respiration can compensate a determined location of the catheter for patient respiration; a signal from the position sensor for patient respiration; and/or the determined position sensor location for patient respiration. In some embodiments, the method can include compensating for the patient respiration by subtracting the respiration compensation for the patient respiration from the position sensor location of the position sensor. By subtracting the respiration compensation for the patient respiration from the position sensor location, the respiration artifact can be removed from the determined location of the position sensor disposed on the catheter. As such, a location and/or movement of the position sensor can be determined, which takes into account the respiration artifact. Thus, an accurate location of the catheter, which is free of the respiration artifacts, can be determined. Although subtraction of the respiration compensation from the position sensor location is discussed, alternatively, and or in addition, the respiration compensation can be subtracted from the determined location of the catheter and/or a signal from the position sensor. Accordingly, a respiration artifact that is present in the determined location of the catheter, a signal from the position sensor, and/or position sensor location can be remove.

As further discussed above, where the method includes determining a second weighted patient reference sensor location, the compensation for patient respiration can be determined based on both the first weighted patient reference sensor location and the second weighted patient reference sensor location. Based on the weights of the first patient reference sensor location and the second patient reference sensor location, the effect of each weight can be varied, based on how closely the first and second patient reference sensor locations track the position sensor. Accordingly, the compensation for the patient respiration based on the first and second weighted patient reference sensor locations can be subtracted from the position sensor location of the position sensor, in order to compensate for the patient respiration.

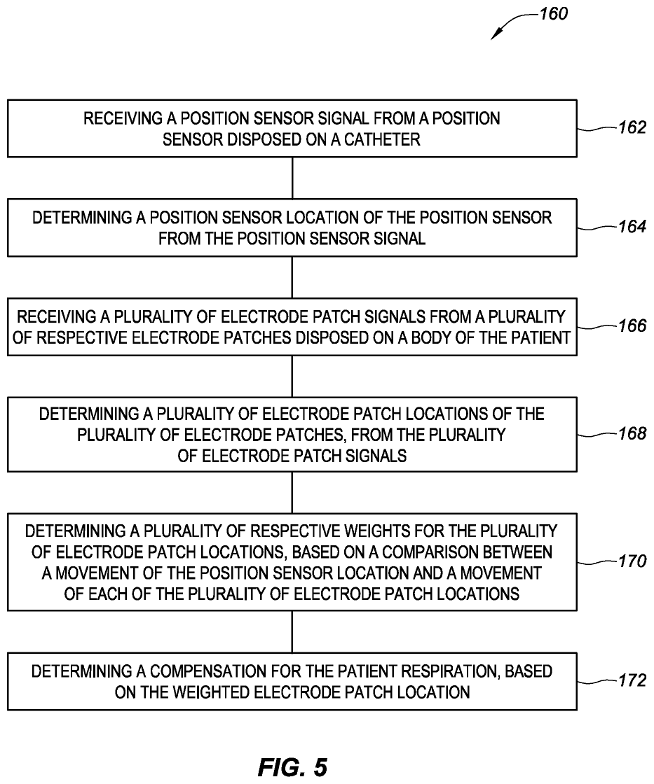

In some embodiments, the method can include compensating for the patient respiration using signals received from one or more electrode patches. For example, in some embodiments, the compensation for patient respiration can be determined based on both the received one or more patient reference sensor signals and one or more electrode patch signals. In some embodiments, where both patient reference sensor signals and electrode patch signals are used in the determination of the compensation for patient respiration, the zero average for the patient reference sensor signals may not be determined. In an example, instead of determining the zero average for the patient reference sensor signals, a high pass filter can be applied to the patient reference sensor signals. For instance, a high pass filter can be applied to the patient reference sensor signals to exclude patient reference sensor signals that are outliers. In some embodiments, the high pass filter can be applied to locations of the patient reference sensor, after processing of the patient reference sensor signal has been completed and the location of the patient reference sensor has been determined. Further discussion with respect to determination of the compensation for patient respiration using electrode patches is provided with reference to FIG. 5.

As discussed, in some embodiments, a signal can be received from a patient reference sensor and from an electrode patch. In some embodiments, the signal received from the patient reference sensor can be magnetically based and the signal received from the electrode patch can be impedance based. Generally, a greater precision can be obtained through using a signal received from the patient reference sensor to determine a compensation for patient respiration. For example, magnetic based position sensors are not subject to shift and drift, like impedance based position sensors. However, in some instances, the signal received from the magnetic patient reference sensor can be subject to distortion. For example, the signal received from the magnetic patient reference sensor can be subject to a metallic distortion in some embodiments, where a component associated with the catheterization laboratory is moved within proximity of a magnetic field in which the magnetic patient reference sensor is disposed and/or within proximity of the magnetic patient reference sensor, itself. Accordingly, in some embodiments of the present disclosure, the method can include determining whether the patient reference signal from the magnetic patient reference sensor is valid. In some embodiments, the method can include determining whether the patient reference signal from the magnetic patient reference sensor is valid, based on whether the magnetic patient reference sensor has been subjected to a metallic distortion. In some embodiments, a determination of magnetic field distortion (e.g., metallic distortion) can be made, as further discussed in WO 2017/130135, which is incorporated by reference as though fully set forth herein.

In some embodiments, if a distorted magnetic patient reference sensor signal is used in determining the compensation for patient respiration, the compensation can be incorrect because the signal has been distorted and is thus inaccurate. Accordingly, embodiments of the present disclosure can exclude the distorted magnetic position reference sensor signal for use in determining the compensation for patient respiration. In lieu of using the magnetic position reference sensor signal, a signal can be received from an electrode patch disposed on the patient. Using the signal received from the electrode patch, as further discussed herein with respect to FIG. 5, can result in a more accurate determination of the compensation for patient respiration, because the signal received form the electrode patch is not subject to the distortion in the magnetic field. Thus, in some embodiments, the method can include determining the compensation for the patient respiration, based on the signal received from the electrode patch.

In some embodiments, even though the signal received from the electrode patch can be used for determining the compensation for patient respiration when the signal received from the magnetic patient reference sensor is invalid; the signal from the magnetic patient reference sensor can still be received and monitored. For example, the signal from the magnetic patient reference sensor can be monitored to determine whether distortion remains in the signal received from the magnetic patient reference sensor. In some embodiments, if the signal received from the magnetic patient reference sensor becomes valid and is thus no longer distorted, the signal received from the magnetic patient reference sensor can be used in lieu of the signal received from the electrode patch.

FIG. 5 depicts an alternate method flow diagram 160 for performing patient respiration compensation, in accordance with embodiments of the present disclosure. As previously discussed, the method 160 can include receiving 162 a position sensor signal from a position sensor disposed on a catheter. In some embodiments, the method 160 can include determining 164 a position sensor location of the position sensor from the position sensor signal. The position sensor signal can be obtained from a magnetic position sensor in some embodiments and/or an electrode disposed on the catheter. In an embodiment where signals are received from both a magnetic position sensor and an electrode disposed on the catheter, the signals can be filtered in some embodiments. For example, the signals can be filtered using an Extended Kalman Filter, in some embodiments.

The measurements obtained through the magnetic position sensor can be more accurate than those obtained through the electrode disposed on the catheter. The Extended Kalman Filter can use a series of catheter positions observed over time, determined from positions obtained through the magnetic position sensor and the electrode, to produce estimates of catheter positions that tend to be more accurate than those based on a single measurement alone.

In some embodiments, the method 160 can include receiving 166 a plurality of electrode patch signals from a plurality of respective electrode patches disposed on a body of the patient. In some embodiments, the electrode patches can be disposed on a patient's body in order to generate an electrical field in the chest of the patient, such that the electrical field can be sensed by an electrode disposed on a catheter that has been inserted into the patient's body. Based on a particular signal sensed by the electrode disposed on the catheter, a position of the electrode and thus the catheter can be determined.

In some embodiments, the method 160 can include determining 168 a plurality of electrode patch locations of the plurality of electrode patches, from the plurality of electrode patch signals. In an example, the electrode patches can generate the electrical field, but can also act as a sensor by generating a signal responsive to locations of the electrode patches in the electrical field. In embodiments where the electrode patches are disposed on a chest of the patient, the location of the electrode patches can be used for determining a compensation for patient respiration. In some embodiments, one or more magnetically based patient reference sensors can also be disposed on a body of a patient, and position signals can be obtained from the magnetically based patient reference sensors, as previously discussed herein. In some embodiments, the compensation for patient respiration can be determined using both the locations of the electrode patches and the locations of the magnetically based patient reference sensors. In embodiments of the present disclosure that compensate for patient respiration using both the locations of the electrode patches and the magnetically based patient reference sensors, a filter can be applied to the locations received from the electrode patches and the magnetically based patient reference sensors. In some embodiments, the filter can be applied to the locations received from the magnetically based patient reference sensors, rather than determining an average value of the signal received from the magnetically based patient reference sensors.

In some embodiments, a compensation for patient respiration can be solved for via the following equation:

C=P·W=Σⱼ₌₁^{J}W^{j}P^{j}

where C (Cᵢ) is the compensation to be subtracted from the location of the position sensor disposed on the catheter, which can be impedance based and/or magnetically based. In some embodiments, P^{j}, j=1, . . . , J, can be the filtered electrode patch and patient reference sensor signals. In some embodiments, the method 160 can include determining 168 a plurality of electrode patch locations of the plurality of electrode patches, from the plurality of electrode patch signals. In an example, for 36 electrode patch signals and one patient reference sensor signal, J would be J=36+1=37 signals. In an embodiment where the patient reference sensor signal is not used, for example, where the patient reference sensor signal is invalid due to a magnetic disturbance, then J=36 signals. Each signal P^{j}, can be a vector

P^{j}=Pᵢ^{j}

where i is a sample index.

In some embodiments, the method 160 can include determining 172 a plurality of respective weights for the plurality of electrode patch locations, based on a comparison between a movement of the position sensor location and a movement of each of the plurality of electrode patch locations. As previously discussed, as a correlation between the position sensor location and a particular one of the electrode patch locations increases, a weight assigned to the particular electrode patch location can be increased. Furthermore, as a correlation between the position sensor location and a particular one of the electrode patch locations decreases, a weight assigned to the particular electrode patch location can be decreased. The weights W^{j}, j=1, . . . , J, are coefficients and can be calculated as the solution of the following optimization problem:

$${‖ x - P \cdot W ‖}^{2} = {\sum }_{i} {( {x}_{i} - P \cdot W )}^{2} = {\sum }_{i} {( {x}_{i} - {\sum }_{j = 1}^{J} {W}^{j} {P}_{i}^{j} )}^{2} \to min$$

To solve for the above referenced optimization problem, a singular value decomposition of the matrix

P=(Pᵢ^{j}),

where

P=U·S·V^{T},

with U and V being orthogonal matrices, and S being a diagonal matrix. We denote by sⱼ, j=1, . . . , J, the diagonal entries of S, s₁≥s₂≥ . . . ≥s_{J}. Let \tilde{S}⁻¹ be the pseudoinverse of S, the J×J diagonal matrix with these values on the main diagonal: s₁⁻¹, s₂⁻¹, . . . , sₖ⁻¹, 0, 0, . . . , 0, with 1<k<J. k is chosen so that all the singular values sₖ₊₁, sₖ₊₂, . . . , s_{J} represent the calculation noise, for example, k=8. The solution to the above referenced optimization problem is given by the following:

W=V·\tilde{S}⁻¹·U·x.

In some embodiments, the method 160 can include determining 172 a compensation for the patient respiration, based on the weighted electrode patch location and the weighted reference sensor location. The compensation for patient respiration, C (Cᵢ), can be subtracted from the location of the position sensor disposed on the catheter, which can be impedance based and/or magnetically based.

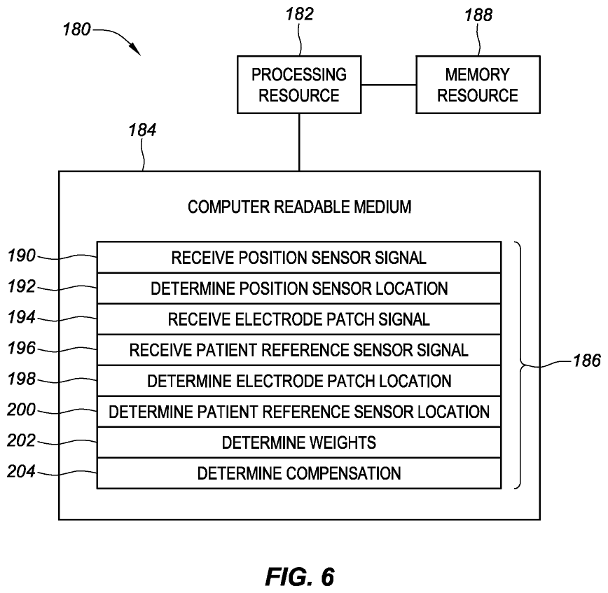

FIG. 6 depicts a system for performing patient respiration compensation, in accordance with embodiments of the present disclosure. Embodiments of the present disclosure can include a computer system 180, as also discussed in relation to FIG. 1, which can utilize software, hardware, firmware, and/or logic to perform a number of functions. The computer system 180 can include a number of remote computing devices, in some embodiments.

The computer system 180 can be a combination of hardware and program instructions configured to perform a number of functions. The hardware, for example, can include one or more processing resources 182, computer readable medium (CRM) 184, etc. The program instructions (e.g., computer-readable instructions (CRI) 186) can include instructions stored on CRM 184 and executable by the processing resource 182 to implement a desired function (e.g., determine a compensation for the patient respiration, based on the weighted electrode patch location and the weighted reference sensor location, etc.). The CRI 186 can also be stored in remote memory managed by a server and represent an installation package that can be downloaded, installed, and executed. The computer system 180 can include memory resources 188, and the processing resources 182 can be coupled to the memory resources 188.

Processing resources 182 can execute CRI 186, which can be stored on an internal or external non-transitory CRM 184. The processing resources 182 can execute CRI 186 to perform various functions, including the functions described with respect to FIG. 1 to FIG. 6.

A number of modules 190, 192, 194, 196, 198, 200, 202, 204 can be sub-modules or other modules. For example, the determine weights module 202 and the determine compensation module 204 can be sub-modules and/or contained within a single module. Furthermore, the number of modules 190, 192, 194, 196, 198, 200, 202, 204 can comprise individual modules separate and distinct from one another.

A receive position sensor signal module 190 can comprise CRI 186 and can be executed by the processing resource 182 to receive a position sensor signal from a position sensor disposed on a catheter. In some embodiments, the position sensor signal can be received from a magnetically based position sensor and/or an impedance based position sensor.

A determine position sensor location module 192 can comprise CRI and can be executed by the processing resource 182 to determine a position sensor location of the position sensor from the position sensor signal. The position sensor signal can be subject to a respiration artifact, causing the location of the position sensor to move, even when the catheter on which the position sensor is disposed is stationary with respect to the heart. Embodiments of the present disclosure can compensate for the respiration artifact.

A receive electrode patch signal module 194 can comprise CRI and can be executed by the processing resource 182 to receive an electrode patch signal from an electrode patch disposed on a body of the patient. In some embodiments, the electrode patch can be disposed on the patient externally. For example, the electrode patch can be disposed on a surface of the skin of the patient. A receive patient reference sensor signal module 196 can comprise CRI and can be executed by the processing resource 182 to receive a patient reference sensor signal from a patient reference sensor. The patient reference sensor can be magnetically based, in some embodiments, and can be disposed on a surface of the skin of the patient. In some embodiments, two patient reference sensors can be disposed on a patient, with one of the patient reference sensors being disposed anteriorly with respect to the patient and one of the patient reference sensors being disposed posteriorly with respect to the patient.

A determine electrode patch location module 198 can comprise CRI and can be executed by the processing resource 182 to determine an electrode patch location of the electrode patch, from the electrode patch signal. As discussed, the electrode patch can generate an electrical field in which an impedance based position sensor can be disposed. In some embodiments, the electrode patch can generate a signal responsive to being disposed in an electrical field, which is generated by other electrode patches disposed on the patient. A determine electrode patch location module 200 can comprise CRI and can be executed by the processing resource 182 to determine an electrode patch location of the electrode patch, from the electrode patch signal.

A determine patient reference sensor location module 200 can comprise CRI and can be executed by the processing resource 182 to determine a location of the patient reference sensor, from the patient reference sensor signal. The locations determined from the electrode patch and the patient reference sensor can be used in the determination of the compensation for patient respiration. In some embodiments, as discussed herein, the patient reference sensor location and the electrode patch location can be filtered. In an example, by filtering the patient reference sensor location and the electrode patch location, outliers can be filtered out, resulting in a more accurate representation of patient respiration.

A determine electrode patch location module 202 can comprise CRI and can be executed by the processing resource 182 to determine respective weights for the electrode patch location and the patient reference sensor location, based on a comparison between a movement of the position sensor location with respect to a movement of the electrode patch location and the patient reference sensor location. As discussed herein, the weights can be adjusted for the electrode patch location and the patient reference sensor location, based on a correlation in movement between the position sensor and the external sensors (i.e., electrode patch, patient reference sensor). In some embodiments, as discussed herein, a weight associated with the patient reference sensor can be decreased in response to a determination that the signal received from the patient reference sensor is invalid due to a magnetic distortion. For example, reducing the weight of the patient reference sensor location to zero in response to the patient reference signal being subjected to a magnetic (e.g., metallic) distortion. Decreasing the weight associated with the patient reference sensor can reduce an effect that the patient reference sensor has on the determination of the compensation for patient respiration. As such, a signal can be continuously received from the patient reference sensor and monitored to determine whether the signal is valid. In response to the signal being valid, indicating that there is no or minimal magnetic distortion in the signal, the weight associated with the patient reference sensor can be increased, increasing its effect on the compensation for the patient respiration.

A determine compensation module 204 can comprise CRI and can be executed by the processing resource 182 to determine a compensation for the patient respiration, based on the weighted electrode patch location and the weighted patient reference sensor location. In some embodiments, the compensation for patient respiration can compensate a determined location of the catheter for patient respiration. In some embodiments, the compensation for patient respiration can compensate a signal from the position sensor for patient respiration. In some embodiments, the compensation for patient respiration can compensate the determined position sensor location for patient respiration. In some embodiments, the compensation for patient respiration can be subtracted from the determined location of the catheter, a signal from the position sensor, and/or the determined position sensor location. Accordingly, a respiration artifact that is present in the determined location of the catheter, a signal from the position sensor, and/or position sensor location can be removed.

Embodiments are described herein of various apparatuses, systems, and/or methods. Numerous specific details are set forth to provide a thorough understanding of the overall structure, function, manufacture, and use of the embodiments as described in the specification and depicted in the accompanying drawings. It will be understood by those skilled in the art, however, that the embodiments may be practiced without such specific details. In other instances, well-known operations, components, and elements have not been described in detail so as not to obscure the embodiments described in the specification. Those of ordinary skill in the art will understand that the embodiments described and illustrated herein are non-limiting examples, and thus it can be appreciated that the specific structural and functional details disclosed herein may be representative and do not necessarily limit the scope of the embodiments, the scope of which is defined solely by the appended claims.

Reference throughout the specification to “various embodiments,” “some embodiments,” “one embodiment,” or “an embodiment”, or the like, means that a particular feature, structure, or characteristic described in connection with the embodiment(s) is included in at least one embodiment. Thus, appearances of the phrases “in various embodiments,” “in some embodiments,” “in one embodiment,” or “in an embodiment,” or the like, in places throughout the specification, are not necessarily all referring to the same embodiment. Furthermore, the particular features, structures, or characteristics may be combined in any suitable manner in one or more embodiments. Thus, the particular features, structures, or characteristics illustrated or described in connection with one embodiment may be combined, in whole or in part, with the features, structures, or characteristics of one or more other embodiments without limitation given that such combination is not illogical or non-functional.

It will be appreciated that the terms “proximal” and “distal” may be used throughout the specification with reference to a clinician manipulating one end of an instrument used to treat a patient. The term “proximal” refers to the portion of the instrument closest to the clinician and the term “distal” refers to the portion located furthest from the clinician. It will be further appreciated that for conciseness and clarity, spatial terms such as “vertical,” “horizontal,” “up,” and “down” may be used herein with respect to the illustrated embodiments. However, surgical instruments may be used in many orientations and positions, and these terms are not intended to be limiting and absolute.

Although at least one embodiment for determination of a catheter shape has been described above with a certain degree of particularity, those skilled in the art could make numerous alterations to the disclosed embodiments without departing from the spirit or scope of this disclosure. All directional references (e.g., upper, lower, upward, downward, left, right, leftward, rightward, top, bottom, above, below, vertical, horizontal, clockwise, and counterclockwise) are only used for identification purposes to aid the reader's understanding of the present disclosure, and do not create limitations, particularly as to the position, orientation, or use of the devices. Joinder references (e.g., affixed, attached, coupled, connected, and the like) are to be construed broadly and can include intermediate members between a connection of elements and relative movement between elements. As such, joinder references do not necessarily infer that two elements are directly connected and in fixed relationship to each other. It is intended that all matter contained in the above description or shown in the accompanying drawings shall be interpreted as illustrative only and not limiting. Changes in detail or structure can be made without departing from the spirit of the disclosure as defined in the appended claims.

Any patent, publication, or other disclosure material, in whole or in part, that is said to be incorporated by reference herein is incorporated herein only to the extent that the incorporated materials does not conflict with existing definitions, statements, or other disclosure material set forth in this disclosure. As such, and to the extent necessary, the disclosure as explicitly set forth herein supersedes any conflicting material incorporated herein by reference. Any material, or portion thereof, that is said to be incorporated by reference herein, but which conflicts with existing definitions, statements, or other disclosure material set forth herein will only be incorporated to the extent that no conflict arises between that incorporated material and the existing disclosure material.

What is claimed:

1. A method for performing patient respiration compensation, comprising:
receiving a position sensor signal from a position sensor disposed on a catheter;
determining a position sensor location of the position sensor from the position sensor signal;
receiving a patient reference sensor signal from a patient reference sensor;
determining a patient reference sensor location of the patient reference sensor from the patient reference sensor signal;
determining a weight for the patient reference sensor location, based on a comparison between a movement of the position sensor location and a movement of the patient reference sensor location;
determining an average of the patient reference sensor location; and
determining a compensation for the patient respiration, based on the weighted patient reference sensor location and the average of the patient reference sensor location.

2. The method of claim 1, wherein the weight for the patient reference sensor location is increased as a correlation in the movement between the position sensor location and the patient reference sensor location increases.

3. The method of claim 1, wherein the weight for the patient reference sensor location is decreased as a correlation in movement between the position sensor location and the patient reference sensor location decreases.

4. The method of claim 1, further comprising compensating for the patient respiration by subtracting the compensation for the patient respiration from the position sensor location of the position sensor.

5. The method of claim 1, further comprising:
receiving a second patient reference sensor signal from a second patient reference sensor;
determining a second patient reference sensor location of the second patient reference sensor from the second patient reference sensor signal; and
determining a second weight for the second patient reference sensor location, based on a comparison between a movement of the position sensor location and a movement of the second patient reference sensor location.

6. The method of claim 1, wherein determining the compensation for the patient respiration further includes determining the compensation for the patient respiration based on the second weighted patient reference sensor location.

7. The method of claim 6, further comprising compensating for the patient respiration by subtracting the compensation for the patient respiration from the position sensor location of the position sensor, wherein the compensation for the patient respiration is based on the weighted patient reference sensor location and the second weighted patient reference sensor location.

8. The method of claim 1, wherein receiving the patient reference signal from the patient reference sensor includes receiving the patient reference signal from a magnetic patient reference sensor.

9. The method of claim 8, wherein the method further comprises receiving a signal from an electrode patch.

10. The method of claim 8, the method further comprising determining whether the patient reference signal from the magnetic patient reference sensor is valid.

11. The method of claim 10, wherein determining whether the patient reference signal from the magnetic patient reference sensor is valid includes determining whether the patient reference signal from the magnetic patient reference sensor is valid, based on whether the magnetic patient reference sensor has been subjected to a metallic distortion.

12. The method of claim 11, further comprising determining the compensation for the patient respiration, based on the signal received from the electrode patch.

13. The method of claim 1, wherein receiving the position sensor signal from the position sensor disposed on the catheter includes receiving a magnetic based positioning signal from the position sensor.

14. The method of claim 1, wherein receiving the position sensor signal from the position sensor disposed on the catheter includes receiving an impedance based positioning signal from the position sensor.

15. A method for performing patient respiration compensation, comprising:
receiving a position sensor signal from a position sensor disposed on a catheter;
determining a position sensor location of the position sensor from the position sensor signal;
receiving a plurality of electrode patch signals from a plurality of respective electrode patches disposed on a body of the patient;
determining a plurality of electrode patch locations of the plurality of electrode patches, from the plurality of electrode patch signals;
determining a plurality of respective weights for the plurality of electrode patch locations, based on a comparison between a movement of the position sensor location and a movement of each of the plurality of electrode patch locations; and
determining a compensation for the patient respiration, based on the weighted electrode patch location.

16. The method of claim 15, wherein receiving the position sensor signal from the position sensor includes receiving a position sensor signal from an electrode disposed on the catheter and a magnetic position sensor signal disposed on the catheter.

17. The method of claim 16, further comprising filtering the position sensor signal and the magnetic position sensor signal with an Extended Kalman Filter.

18. The method of claim 15, further comprising filtering the plurality of electrode patch locations.

19. The method of claim 15, further comprising subtracting the compensation for the patient respiration from the location of the position sensor.

20. A system for performing patient respiration compensation, comprising:
a computing device comprising processor resources and memory resources, the memory resources storing computer-readable instructions that, when executed by the processor resources, cause the processor resources to:
receive a position sensor signal from a position sensor disposed on a catheter;
determine a position sensor location of the position sensor from the position sensor signal;
receive an electrode patch signal from an electrode patch disposed on a body of the patient;
receive a patient reference sensor signal from a patient reference sensor;
determine an electrode patch location of the electrode patch, from the electrode patch signal;
determine a patient reference sensor location of the patient reference sensor from the patient reference sensor signal;
determine respective weights for the electrode patch location and the patient reference sensor location, based on a comparison between a movement of the position sensor location with respect to a movement of the electrode patch location and the patient reference sensor location; and
determine a compensation for the patient respiration, based on the weighted electrode patch location and the weighted patient reference sensor location.

21. The system of claim 20, further comprising instructions executable by the processor to filter the patient reference sensor location and the electrode patch location.

22. The system of claim 20, further comprising instructions executable by the processor to reduce the weight of the patient reference sensor location to zero in response to the patient reference signal being subjected to a metallic distortion.
